(19) United States
(12) Patent Application Publication
(10) Pub. No.: US 2012/0009208 A1
(43) Pub. Date: Jan. 12, 2012

(54) USE OF MEMBRANE ATTACK COMPLEX (MAC) AND IMMUNE COMPLEXES (ICs) TO ACTIVATE T-CELLS AND THE GENERATION OF REGULATORY T CELLS

(76) Inventor: Anil Kumar Chauhan, Wildwood, MO (US)

(21) Appl. No.: 13/175,931

(22) Filed: Jul. 4, 2011

Related U.S. Application Data
(60) Provisional application No. 61/399,018, filed on Jul. 6, 2010.

(51) Int. Cl. A61K 39/00; A61P 29/00; A61P 37/02; C12N 5/00

(52) U.S. Cl. 424/184.1; 435/375

(57) ABSTRACT

The invention describes the use of antigen-antibody complexes also known in the field as immune complexes to activate T cells and their potential to differentiate them into effector T cells for use in therapy. It also describes a process to achieve benefit from disrupting this T cell activation mediated by antigen-antibody complex and sublytic MAC of the complement system.

### CROSS-REFERENCE TO RELATED APPLICATIONS

This application claims priority of the following provisional filing.

| Application Number | Filing Date | Patent Number | Issue Date |
|---|---|---|---|
| 61/399,018 | Jul. 06, 2010 | | |

| | |
|---|---|
| Current U.S. Class: | 424; 514; 524; 534; 810 |

1. A. Laurence and J. J. O'Shea, Nat.Immunol. 8, 903-905 (2007).
2. T. Takai, Nat.Rev.Immunol. 2, 580-592 (2002).
3. T. Takai, J.Clin.Immunol. 25, 1-18 (2005).
4. F. Nimmerjahn and J. V. Ravetch, Nat.Rev.Immunol. 8, 34-47 (2008).
5. F. Tedesco et al., J.Exp.Med. 185, 1619-1627 (1997).
6. F. Niculescu, T. Badea, H. Rus, Atherosclerosis 142, 47-56 (1999).
7. S. M. Dashiell, H. Rus, C. L. Koski, Glia 30, 187-198 (2000).
8. M. Fosbrink, F. Niculescu, V. Rus, M. L. Shin, H. Rus, J.Biol.Chem. 281, 19009-19018 (2006).
9. M. Fosbrink, F. Niculescu, H. Rus, Immunol.Res. 31, 37-46 (2005).
10. M. G. Roncarolo and M. Battaglia, Nat.Rev.Immunol. 7, 585-598 (2007).
11. R. Aricha, T. Feferman, S. Fuchs, M. C. Souroujon, J.Immunol. 180, 2132-2139 (2008).
12. J. Bayry, S. Lacroix-Desmazes, S. Dasgupta, M. D. Kazatchkine, S. V. Kaveri, Nat.Rev.Immunol. 8, 1p (2008).
13. A. Gallimore and A. Godkin, Immunology 123, 157-163 (2008).
14. E. Jaeckel, N. Mpofu, N. Saal, M. P. Manns, Horm.Metab Res. 40, 126-136 (2008).
15. E. Toubi, Isr.Med.Assoc.J. 10, 73-76 (2008).
16. O. Joffre et al., Nat.Med. 14, 88-92 (2008).
17. G. Korczak-Kowalska et al., Transplant.Proc. 39, 2721-2723 (2007).
18. R. A. O'Connor and S. M. Anderton, J. Neuroimmunol. 193, 1-11 (2008).
19. M. H. Zheng et al., Expert.Opin.Ther.Targets. 12, 313-326 (2008).
20. R. H. Schabowsky, S. Madireddi, R. Sharma, E. S. Yolcu, H. Shirwan, Curr.Opin.Investig.Drugs 8, 1002-1008 (2007).
21. E. M. Shevach, Nat.Rev.Immunol. 2, 389-400 (2002).
22. S. Sakaguchi, Cell 101, 455-458 (2000).
23. W. Chen et al., J.Exp.Med. 198, 1875-1886 (2003).
24. K. Kretschmer et al., Nat.Immunol. 6, 1219-1227 (2005).
25. J. Lohr, B. Knoechel, A. K. Abbas, Immunol.Rev. 212, 149-162 (2006).
26. T. S. Davidson, R. J. DiPaolo, J. Andersson, E. M. Shevach, J.Immunol. 178, 4022-4026 (2007).
27. M. C. Fantini et al., J.Immunol. 172, 5149-5153 (2004).
28. S. Liang et al., J.Exp.Med. 201, 127-137 (2005).
29. Y. Tohyama and H. Yamamura, J.Biochem. 145, 267-273 (2009).
30. A. Mocsai, J. Ruland, V. L. Tybulewicz, Nat.Rev.Immunol. 10, 387-402 (2010).
31. H. von Boehmer and F. Melchers, Nat.Immunol. 11, 14-20 (2010).
32. J. Zhu, H. Yamane, W. E. Paul, Annu.Rev.Immunol. 28, 445-489 (2010).
33. J. C. Crispin, V. C. Kyttaris, C. Terhorst, G. C. Tsokos, Nat.Rev.Rheumatol. 6, 317-325 (2010).
34. F. Nimmerjahn and J. V. Ravetch, Adv.Immunol. 96, 179-204 (2007).
35. E. Shores et al., J.Immunol. 159, 222-230 (1997).
36. S. M. Hayes and P. E. Love, Immunity. 16, 827-838 (2002).
37. H. Arase et al., J.Exp.Med. 182, 891-895 (1995).
38. E. J. Enyedy et al., Arthritis Rheum. 44, 1114-1121 (2001).
39. S. Krishnan, V. G. Warke, M. P. Nambiar, G. C. Tsokos, D. L. Farber, J.Immunol. 170, 4189-4195 (2003).
40. C. W. Thomson et al., J.Immunol. 177, 2250-2257 (2006).
41. W. B. van den Berg, Curr.Opin.Rheumatol. 9, 221-228 (1997).
42. P. L. van Lent et al., Arthritis Rheum. 43, 740-752 (2000).
43. A. J. Szalai, X. Hu, C. Raman, S. R. Barnum, Eur.J.Immunol. 35, 3487-3492 (2005).
44. T. Tsubata and J. Wienands, Int.Rev.Immunol. 20, 675-678 (2001).
45. N. Gupta and A. L. DeFranco, Semin.Cell Dev.Biol. 18, 616-626 (2007).
46. J. S. Mitchell, O. Kanca, B. W. McIntyre, J.Immunol. 168, 2737-2744 (2002).
47. M. L. Alegre, K. A. Frauwirth, C. B. Thompson, Nat.Rev.Immunol. 1, 220-228 (2001).
48. R. T. Abraham and A. Weiss, Nat.Rev.Immunol. 4, 301-308 (2004).
49. J. V. Ravetch and B. Perussia, J.Exp.Med. 170, 481-497 (1989).

### BACKGROUND OF INVENTION

T cells when presented with peptide-MHC complex by antigen presenting cells (APC) engage the T cell receptor (TCR) resulting in T cell activation. This activation induces proliferation of T cells. These proliferating T cells when presented with appropriate cytokines result in differentiation of naïve T cells into Th1, Th2, Th17, and Treg cells¹.

The immune complexes (IC) bind to receptors for immunoglobulin Fc region (FcRs) that are expressed by leukocytes and play an important role in both promoting and regulating immune and inflammatory responses by activating signaling events²⁻⁴. The ICs are formed in the disease process by association of antibodies against disease associated antigen and they activate the complement system. This complement activation result in the generation of complement by products. One of such product is C5b, which by associating with other proteins of complement system C6, C7, C8 and C9 forms the membrane attack complex (MAC) on cell membrane. This MAC complex is also observed in the plasma and is capable of inducing biological activity in endothelial cells⁵. We observed that when peripheral human naïve CD4⁺ T cells (CD45RA⁺) are treated with ICs purified from disease plasma and non-lytic amount of MAC (defined as the amount of MAC that does not cause cell lysis, hereafter referred to as sublytic MAC), either purified from plasma or assembled from or with purified complement proteins, results in proliferation of these cells. This also causes the phosphorylation of T cell signaling pathway proteins such as ζ-chain, ZAP-70 and Syk. We also observed the expression of low affinity, ICs binding FcγRIII receptors in CD4⁺ T cells.

The lack of the presence of low affinity receptors on T cells has been noted and their presence has been suggested as an open question⁴. When we treated CD4⁺ T cells with ICs and sublytic amount of MAC, we observed phosphorylation of Syk (FIG. 3). Upon phenotyping the CD4⁺ T cells from SLE patient with fluorescently labeled ICs, 5 to 10% of CD4⁺ T cells bound to these labeled ICs compared to up to 4% in normal healthy subjects. This suggested the presence of low affinity FcγR on CD4⁺ T cells.

Treatment of CD4⁺ T cells with sublytic dose of MAC along with purified ICs induced T cell proliferation. Proliferation by sublytic MAC in aortic endothelial cells, smooth muscle cells and Schwann cells has been previously reported⁶⁻⁸. The MAC mediated proliferation is accompanied by phosphorylation of ERK⁹. In primary mesangial cells, we also observed upregulation of ERK1/2 in response to ICs and sublytic MAC. We observed that there was always a requirement for both IC and sublytic MAC to induce a biological response in T cells and mesangial cells. We further observed that the CD4⁺ T cells activated by sublytic MAC and ICs when allowed to grow under polarizing cytokines in this case IL-2 and TGFβ, they showed induced expression of forkhead box P3 (FoxP3) transcription factor and an increase in CD25 expression, a marker of T cell activation, thus demonstrating the development of induced regulatory T cells (iTreg).

Several mechanisms contribute to the tolerance in the periphery to self-antigens; one of such mechanisms is the activity of regulatory T lymphocytes. The naturally occurring FoxP3⁺, CD4⁺, CD25⁺ regulatory T cells develop in thymus and are present in healthy individuals¹⁰. The potential for the use of expanded Treg cells for therapeutic interventions in several diseases is currently being investigated¹¹⁻²⁰.

Mice treated with regulatory CD4⁺CD25⁺Foxp3⁺ T cells stimulated in vitro with alloantigens are known to induce long-term tolerance against bone marrow and subsequent skin and cardiac allografts. Treg cells specific for both directly and indirectly presented alloantigens have been shown to prevent both acute and chronic rejections. iTreg cells stimulated with appropriate antigen can be used for inducing lifelong immunological tolerance¹⁶.

Activated CD4⁺ T cells expressing CD25 under neutral stimulation of TCR do not show any suppressive ability^{21;22}. iTreg with FoxP3 expression can also be developed from naïve CD25⁻, FoxP3⁻, CD4⁺ T cells in response to antigen presented with MHC recognition and by driving their differentiation with TGF-β²³⁻²⁵. Signal supporting Foxp3 induction by TGF-β arise from the engagement of TCR and a co-stimulation from CD28 and IL-2R²⁴⁻²⁸.

All the methods that are currently used for the generation of ex vivo iTreg cells use CD4⁺ cells that are stimulated in vitro using anti-CD3 and anti-CD28 antibodies in the presence of TGF-β and IL-2. The obtained cells express high levels of CD25, CTLA-4, and Foxp3, and are capable of suppressing in vitro proliferation of T cells in response to auto-antigens and are considered as potential agents for the treatment of inflammatory disorders¹¹

We describe the use of ICs that can be purified from plasma of patients and MAC that can either be purchased commercially or can be purified from normal plasma that is activated with Zymogen A, as immunomodulators. Use of purified ICs along with sublytic MAC can be used to differentiate naïve CD4⁺ T cells, purified from human plasma into CD4⁺ CD25⁺ and FoxP⁺ cells, an effector T cell phenotype; in this instance iTregs. We also show that sublytic doses of MAC with ICs when used along with the anti-CD3, show more potent activation of TCR signal and that this signal is sufficient for increasing the iTreg cells expansion by four fold over the original iTreg cell population (FIG. 5). The anti-CD3 antibody alone is insufficient to generate such response (FIG. 5).

Spleen tyrosine kinase (Syk) is a non-receptor tyrosine kinase, expressed by hematopoietic cells that play a crucial role in adaptive immunity²⁹. Syk activation is important for cellular adhesion, vascular development, osteoclast maturation, and innate immune recognition. Syk activation, target pathways such as the CARD9-BCL-10-MALT1 and the NLRP3 inflammasome³⁹. In autoimmunity, altered T lymphocyte responses are observed^{31;32}. In systemic lupus erythematosus (SLE) patients, enhanced T cell antigen receptor (TCR) signaling is shown to contribute to the disease pathogenesis³³. ICs induce phosphorylation of the FcRγ chain, the signaling subunit of Fc receptors (FcR). This triggers Syk phosphorylation in B cells, which is important for B cell activation³⁴. Interestingly, FcRγ chain in T cells associates with the ζ-chain of the TCR and can independently support the development of the peripheral T cells. The FcRγ chain alone is also sufficient to support the development of peripheral T cells in mice lacking endogenous TCR ζ-chain³⁵.

FcRγ chain containing TCR complexes are present in activated γδ⁺ T cells, NK-like T (NKT) cells, SLE-T cells, and in certain populations of human T effector cells³⁶⁻³⁹. An association of FcRγ chain with the TCR complex is also observed in TCRαβ⁺CD4⁻CD8⁻ double-negative regulatory T cells⁴⁰. In these cells, TCR ligation results in the phosphorylation of FcRγ chain and Syk, which is necessary for their suppressive activity⁴⁰. The TCR of CD4⁺ T effector cells showed association of FcRγ chain with Syk³⁹. Such events are also observed in antigen induced arthritis (AIA), a chronic arthritis regulated by ICs and T cells⁴¹. In AIA, inflammation and cartilage erosion is dependent on FcRγ chain mediated signaling⁴². Also, for full development of experimental autoimmune encephalomyelitis (EAE), expression of FcRγ chain in conjunction with TCR/CD3 complex by γδ T cells is required⁴³. The presence of ICs is also documented in these disease conditions.

We observed that sublytic MAC synergistically in the presence of ICs enhances the Syk phosphorylation in T cells. We also observed aggregation of the membrane rafts (MRs) in cells treated with sublytic amount of MAC assembled on the T cell membrane using purified complement proteins. The MRs are membrane structures that are crucial for cell signaling involving Lyn, Syk, and Btk kinases in activated lymphocytic cells^{44;45}. In T cells, signaling molecules such as Syk associate with MR. The lateral diffusion of MRs allows protein interaction and initiating signaling⁴⁶. Such events are also known to be facilitated by co-stimulatory molecule CD28⁴⁷.

### BRIEF SUMMARY OF THE INVENTION

The invention is described in the field of immune modulation. Such modulation of immune responses using ICs and MAC, sometime also referred to as terminal complement complex (TCC) when present in systemic circulation can promote generation of effector T cells. A synergistic action of both these protein immune reactants is required for such events. Thus inhibition of development of harmful T effector cells from IC and MAC can be used to generate a favorable outcome during the disease process. Alternately, the iTreg can be generated using ICs and sublytic MAC and subsequently used for the treatment of inflammatory autoimmune disorders by adoptive transfer. Such process can be used for development of immunotherapeutics for inflammatory autoimmune diseases.

### BRIEF DESCRIPTION OF THE SEVERAL VIEWS OF THE DRAWING

“This patent or application file contains at least one drawing executed in color. Copies of this patent or patent application publication with color drawing(s) will be provided by the Office upon request and payment of the necessary fee.”

FIG. 1 establishes the presence of receptors that bind to ICs on T cells.

FIG. 1A:

Competitive Inhibition of Alexa Fluor—488-AHG binding with anti-FcμRIIIb and anti-FcγRI antibodies on human CD4⁺ T cells. (A) Increased intensity of fluorochrome label bound to cells, treated with increasing amount of fluorochrome labeled AHG. (B) Inhibition of AHG binding with anti-FcγRIIIb antibody. Binding of labeled AHG to CD4⁺ T cells was inhibited by 50% using 5 μg of anti-FcγRIIIb antibody. (C) Inhibition of labeled AHG binding with anti-FcγR1 antibody. Inhibition of binding of AHG to human CD4⁺ T cells was not observed using anti-FcγRI antibody at a conc. of 10 and 20 μg respectively.

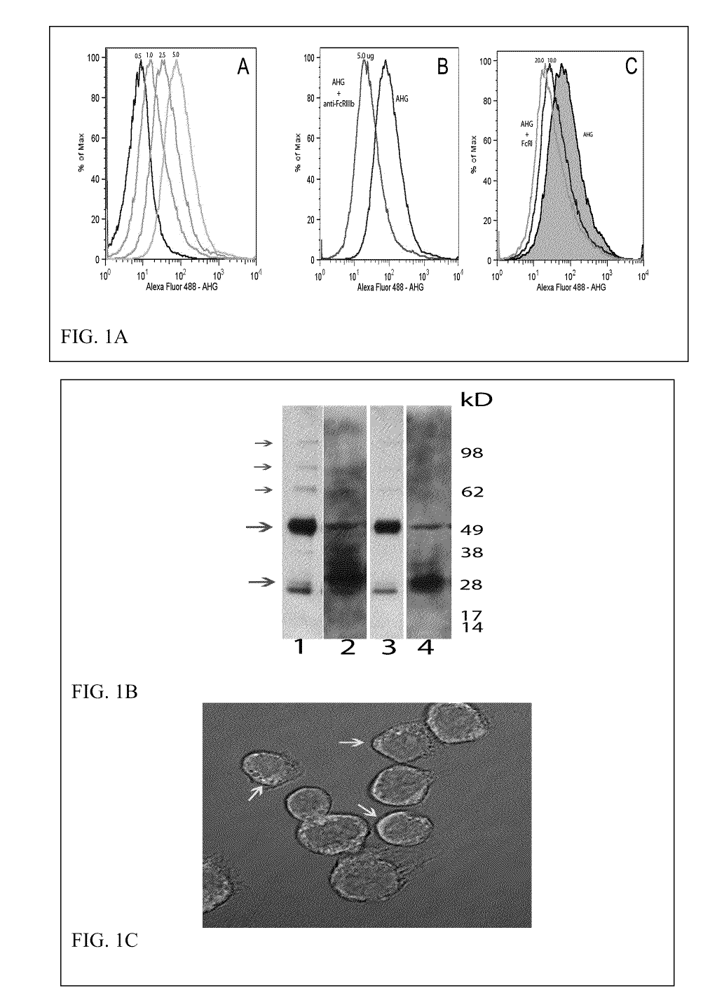

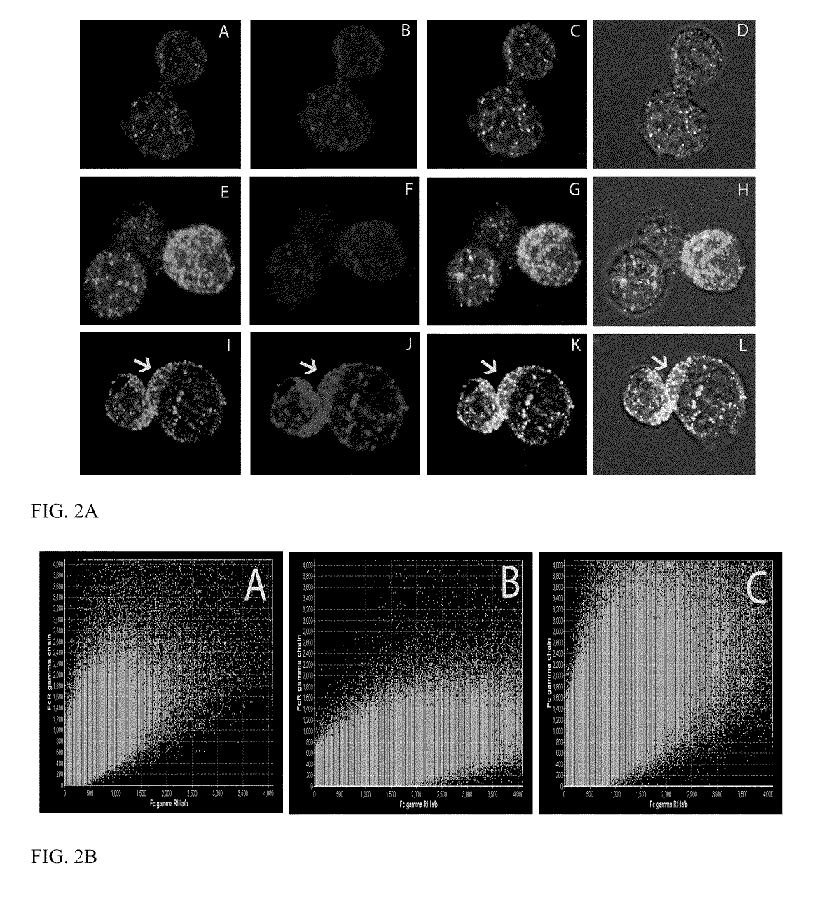

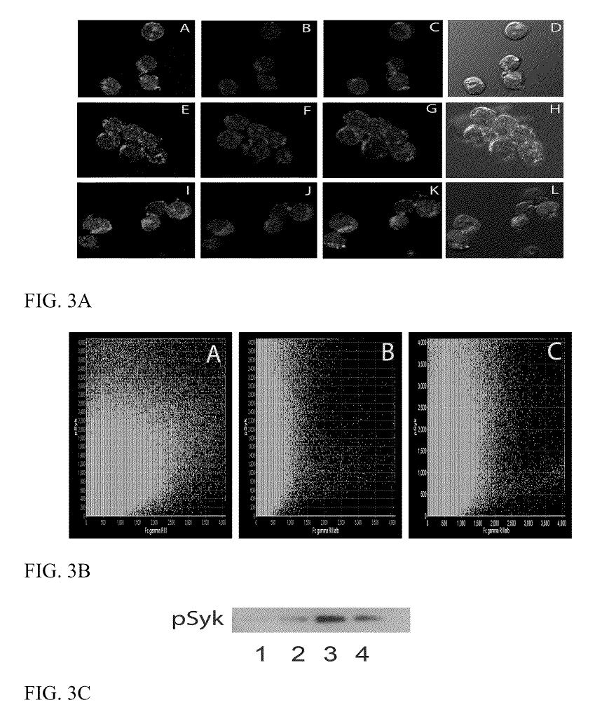

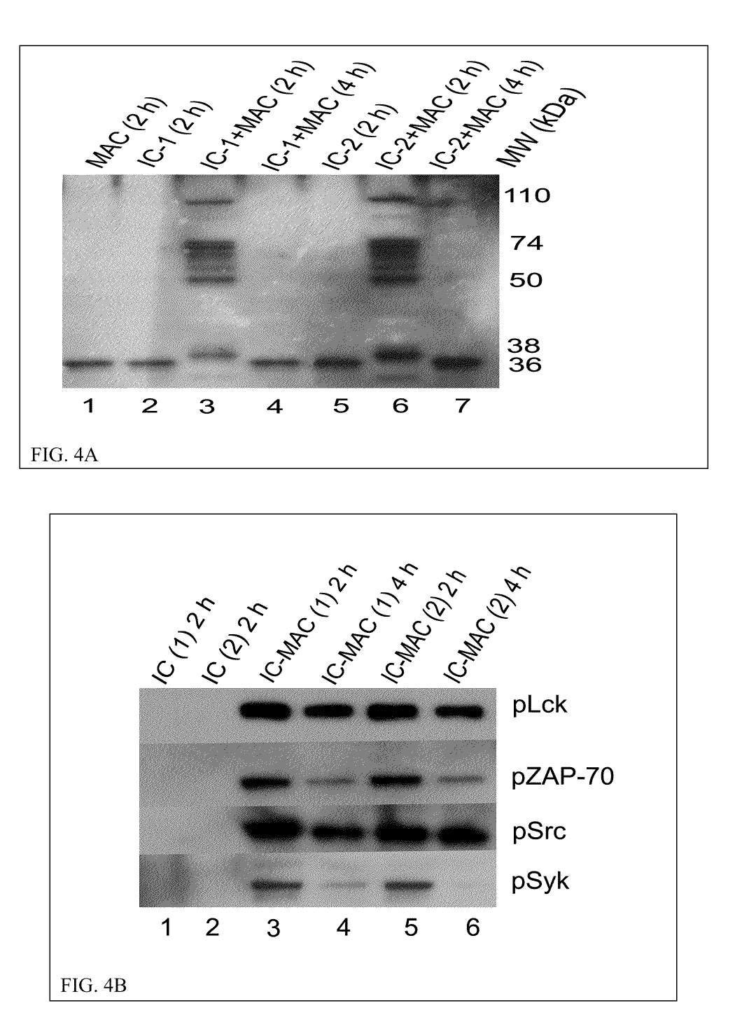

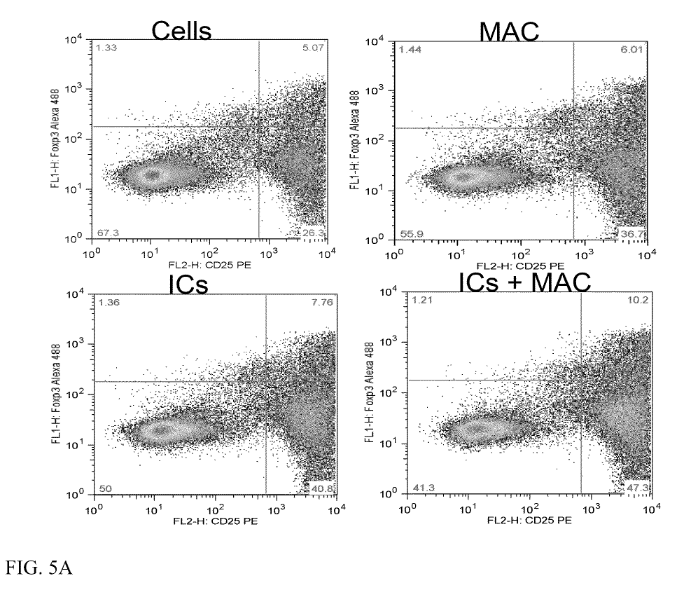

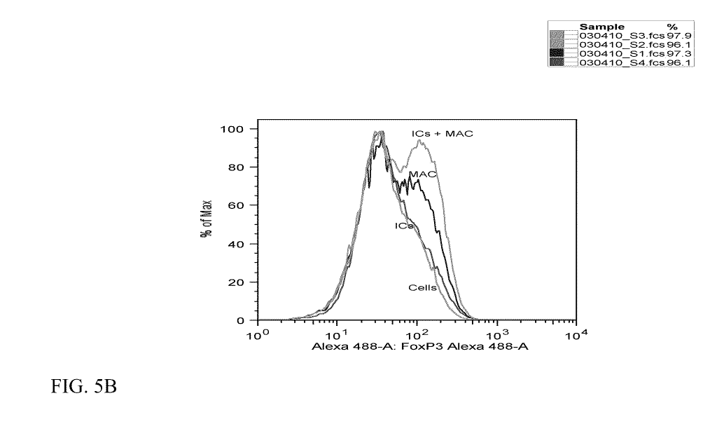

FIG. 1B:

SDS-PAGE analysis of immunoprecipitates generated using a monoclonal anti-FcγRIIIa/b from CD4⁺ T cells (lane 1) and Jurkat cells (lane 3). Silver staining of proteins CD4⁺ T cells (lane 1) and Jurkat cells (lane 3). Western blot analysis of immunoprecipitates probed with anti-FcγRIIIa/b, CD4⁺ T cells (lane 2) and Jurkat cells (lane 4). Western blot show the presence of 26 kD to 29 kD proteins indicating the presence of FcγRIIIa/b protein.

FIG. 1C:

Presence of FcγRIIIa/b protein analyzed with confocal microscopy in human CD4⁺ T cells. A human CD4⁺ T cells showing the staining for FcγRIIIa/b receptor using differential interference contrast (DIC). Red staining underneath the cell membrane represents the receptor protein.

FIG. 2 establishes that when T cells are treated with ICs and MAC, this triggers activation of Fc receptor mediated immune responses, since the FcRγ chain recruits to membrane receptor.

FIG. 2A:

Figure shows that in response to ICs and sublytic MAC treatment FcRγ chain co-localizes with membrane FcγRIIIa/b. Co-localization of FcγRIIIa/b and FcRγ chain in untreated CD4⁺ T cells (panels ABCD), cell treated with ICs purified from SLE patient (panels EFGH), and cells treated together with ICs and sublytic MAC (panels IJKL). AEI stained for FcγRIIIa/b; BFJ stained for FcRγ chain, CGK co-localization of FcγRIIIa/b and FcRγ chain; DHL co-localization of FcγRIIIa/b and FcRγ chain on differential interefrence ontrast (DIC) image.

FIG. 2B:

Co-localization analysis using Olympus FV-1000 microscope software for membrane FcγRIIIa/b and FcRγ chain in CD4⁺ T cells. Distribution of FcγRIIIa/b and FcRγ chain in CD4⁺ T cells untreated (A), treated with ICs (B) and together ICs with sublytic MAC(C) in total z-series images. Y-axis displays FcRγ chain and X-axis displays FcγRIIIa/b.

FIG. 3 establishes that the T cells treated with ICs and MAC show activation of Syk signaling pathway.

FIG. 3A

Human CD4⁺ T cells treated with IC and sublytic MAC show association of pSyk with FcγRIIIa/b. Confocal images of human CD4⁺ T cells showing co-localization of FcγRIIIa/b and pSyk. Untreated cells (ABCD); treated with SLE-ICs (EFGH); and SLE-ICs and sublytic MAC (IJKL). AEI stained for FcγRIIIa/b; BFJ for pSyk, CGK FcγRIIIa/b+pSyk; DHL shown on DIC image.

FIG. 3B

Co-localization distribution analysis of FcγRIIIA/B and pSyk obtained using Olympus FV-1000 software of images shown in FIG. 3A. Untreated cells (A); treated with ICs (B); ICs and sublytic MAC(C). Y-axis show pSyk and X-axis shows FcγRIIIA/B. ICs in the presence of sublytic MAC show enhanced association of pSyk with FcγRIII.

FIG. 3C

Treatment of CD4⁺ T cells with ICs and sublytic MAC show Syk phopshorylation. Anti-FcγRIIIa/b immunoprecipitates analyzed for Syk phosphorylation. Untreated cells (lane 1), sublytic MAC (lane 2), sublytic MAC and ICs (lane 3), and ICs treated (lane 4) cells. Cells treated with ICs and sublytic MAC show phosphorylation of Syk. Blots were prepared with 5 μg of protein, after western analysis the blots were stained, scanned and analyzed with Image J (NIH, USA) for uniform protein loading.

FIG. 4 shows activation of downstream signaling event of T cells receptor upon treatment with ICs and MAC

FIG. 4A:

Analysis of protein phosphorylation in CD4⁺ T cells (Jurkat cells) treated with ICs, sublytic MAC and both ICs with sublytic MAC. Western blot analysis of cell lysates probed with an anti-PY 20 antibody in cells treated SLE-IC purified from two patients marked (1) and (2). Cells lysates prepared after treatments as shown on the top of lanes. Cells treated with SLE-IC together with sublytic MAC lanes 3 and 6 show phosphorylation of several proteins from 50 to 74 kDa. Proteins at 36, 38 and 110 kDa were also phosphorylated.

FIG. 4B:

Characterization of phosphorylated proteins in Jurkat cells with ICs and sublytic MAC. Western blot analysis of cells treated as described in FIG. 9). Blots probed for phosphorylated Lck, ZAP-70, Src, and Syk. All four proteins show phosphorylation in response to IC and MAC together. Cells treated for 2 h showed enhanced phosphorylation in comparison to 4 h interval.

FIG. 5 show that the T cells activated by ICs and MAC show the potential to get converted into effector T cell population, in this experiment into iTregs.

FIG. 5A:

Generation of T effector cells, after activating them with ICs and sublytic amount of MAC. Conversion of peripheral naïve CD4⁺ T cells into CD25⁺FoxP3⁺ T cells post stimulation with SLE-ICs and sublytic MAC. The CD25⁺FoxP3⁺ population of cells increased from 5.07 to 10.2% in cells treated with SLE-ICs and sublytic MAC. Y-axis shows FoxP3 and X-axis show staining for CD25.

FIG. 5B:

Figure is a histogram showing generation of CD25⁺FoxP3⁺ T cells. When human purified peripheral naïve CD4⁺ T cells were treated with sublytic dose of MAC alone at a concentration of 2.5 μg; IC alone at a concentration of 1 μg, or combination of both ICs and sublytic MAC, in the presence of anti-CD3 at a concentration of 0.5 μg for each one million cells, for a time period of 2 h. The cells were cultured for five days in the presence of 100 ng of TGFβ and 20 IU of IL-2. These cells were phenotyped with flowcytometery for CD25 and FoxP3. On X-axis are the cells stained with anti-FoxP3-Alexa 488 antibody. The CD4⁺CD25^{high} cells treated with IC and sublytic MAC together showed generation of iTregs marked by expression of FoxP3 transcription factor, seen as a second peak.

### DETAILED DESCRIPTION OF THE INVENTION

### General Description

In this invention first, we establish the binding of aggregated human γ-globulin (AHG) used as a model IC to human naïve CD4⁺ T cells. We establish that this binding on human naïve CD4⁺ CD45RA⁺ T cells is mediated by low affinity FcγRIII receptor. The human naïve CD4⁺ T cells or Jurkat cells, a thymoma cell line used to study T cell activation⁴⁸ when treated with sublytic MAC and ICs demonstrate activation of downstream TCR signaling pathway. Thereafter, we established that naïve T cells when treated with sublytic MAC with IC alone and/or in presence of anti-CD3 with appropriate cytokine triggers them to differentiate into T effector cells in this case CD25⁺FoxP3⁺ regulatory T cells.

### Binding of Aggregated Human γ-Globulin to CD4⁺ T Cells:

To explore the presence of FcRs on T cells, we investigated the binding of Alexa Fluor® 488 labeled AHG to these cells. We used human naïve CD4⁺ T cells to establish the presence of FcγRIII receptor. The cell population used for these experiments was 95% positive for CD3 and CD4⁺ by flow analysis. Over 97% of the cells were also positive for CD45RA, a marker expressed on human naïve T cells. In a flow cytometric binding analysis a total of 1×10⁶ T cells treated with 0.5 to 5 μg of Alexa Fluor® 488 labeled AHG showed linear binding. The proportionate increase of AHG concentration to treat the cells showed increase in the cell numbers that bound to AHG and dye, measured by mean fluorescent intensity (FIG. 1A, left panel). These results confirmed that these cells demonstrated receptors that bound ICs.

Inhibition of AHG Binding with Anti-FcγRIIIb:

To further characterize the nature and specificity of AHG binding to CD4⁺ T cells, we utilized a recently developed specific blocking antibody against FcγRIIIb (purchased from R & D systems, USA). Using 5 μg of labeled AHG for 1×10⁶ cells, at a concentration of 5 μg/ml of blocking antibody, we observed a 50% inhibition of the labeled AHG binding. This suggests that under these conditions, the AHG binding to T cells was mediated by FcγRIIIb (FIG. 1A, middle panel). At the same ratio of the cells to labeled AHG amount, the anti-FcγRI antibodies at concentrations of 10 and 20 μg/ml, did not inhibit AHG binding (FIG. 1C, right panel). The rabbit F(ab)₂ fragment used as a control at 10 μg/ml also did not inhibit the labeled AHG binding. In addition, we also tested sublytic MAC alone at 5 μg/ml; at this concentration inhibition of labeled AHG was not seen. Similar results of AHG binding inhibition were obtained using a monoclonal antibody that recognizes FcγRIIIa/b, both receptors.

FcγRIII Immunoprecipitation from Jurkat and Human CD⁺ T Cells:

To further characterize the nature of the protein mediating the binding of AHG to CD4⁺ T cells, we generated the immunoprecipitates from the CD4⁺ naïve T cells using monoclonal anti-FcγRIIIa/b. In addition to heavy and light chain bands of mouse immunoglobulin, these precipitates showed the presence of a protein band that migrated at the molecular weight of 29 kD that corresponds to earlier reported molecular mass of FcγRIII (FIG. 1B)². In NK cells and PMN, FcγRIII migrate as a broad band of apparent mass of 50-70 kD⁴⁹. The FcγRIIIa consists of 254 amino acids with a predicted mass of 29 kD (Accession no. P08637-1) and FcRIIIb consists of 233 AA with predicted mass of 26 kD (Accession no. o75015-1). In addition, bands at 72, 98 and 130 kD were also observed. The same gel migration pattern of proteins was observed from the purified naïve human CD4⁺ as well as Jurkat cells. Treatment of the cells by MAC, ovalbumin-antiovalbumin immune complexes (Ova-ICs), SLE-ICs or MAC with ICs did not alter the protein profile. Both FcγRIIIa and FcγRIIIb proteins are encoded by two different but nearly identical genes. The Phe-203 in FCGR3A determines the transmembrane region, while Ser-203 in FCGR3B determines the GPI-anchoring. The presence of appropriate molecular mass proteins is suggestive of the presence of the low affinity Fc receptor on human naïve CD4⁺ T cells (FIG. 1B). This established the presence of ICs binding receptors on CD4⁺ T cells.

Cellular Distribution of FcγRIIIa/b and association to FcRγ chain:

Confocal images of cell stained using anti-FcγIIIa/b antibodies in both naïve CD4⁺ T cells and Jurkat cells demonstrated peripheral membrane distribution (FIG. 1C). This could also be observed in cells stained without permeabilization. To further investigate the recruitment of FcRγ—chain, cells after treatment with ICs alone or in combination with sublytic MAC were stained for FcγRIIIa/b antibody and FcRγ chain antibody. For FcRγ-chain staining the cells were permeabilized before staining. The co-localization of FcγRIIIa/b and FcRγ chain was carried out using the Olympus FV-1000 software. This analysis revealed recruitment of FcRγ chain with FcγRIIIa/b upon treatment of the cells with ICs (FIG. 2A) The treatment of these cells with ICs together with sublytic MAC appeared to trigger both the co-localization and capping of both proteins (FIG. 2B). This further suggests the presence of FcγRIIIa and an active recruitment of FcRγ chain upon ligation with its known ligand ICs in CD4⁺ T cells. In addition, phosphorylation of Syk in response to ICs also suggests the presence of FcγRIIIa as Syk associates to the phosphorylated FcRγ chain (FIG. 3).

ICs and Sublytic MAC Treatment Recruits Phosphorylated Syk with FcγRIIIa/b in CD4⁺ T Cells:

As we observed the Syk phosphorylation in our Western blot analysis, we further investigated the role of ICs in the activation of Syk and recruitment of FcRγ chain by ligation of membrane Fc receptors (FcRs) with ICs in T cells. In one experiment we analyzed the co-localization of phosphorylated Syk (pSyk) with FcγRIIIA/B on membrane in cells treated with ICs and sublytic MAC. The T cells were stained using the anti-pSyk and a monoclonal antibody recognizing FcγRIII both A and β isoforms. The confocal examination of these cells showed that upon IC treatment, pSyk moved to the sites of FcγRIIIA/B staining (FIG. 3A). Scatter plot for localization of pSyk and FcγRIIIA/B staining obtained for all z-series sections by using co-localization software confirmed that the treatment phosphorylated Syk that moved to the location of FcγRIIIa/b in the cells (Olympus FV-1000) (FIG. 3B). The treatment of cells with ICs alone demonstrated a shift towards the stronger staining for pSyk along y-axis (FIG. 3A panel B). This shift was further enhanced in the presence of sublytic MAC as observed by an increase in the intensity of pSyk scatter along y-axis (FIG. 3B panel C). Sublytic MAC alone was not sufficient to trigger this event. Our results show that T cells in response to their treatment with ICs and sublytic MAC result in co-localization of pSyk with FcγRIII (accepted for publication in Journal of Clinical and Experimental Immunology).

Phosphorylation of Syk in Response to Treatment with ICs and Sublytic Mac:

Syk is critical for ITAM mediated signaling in immune cells that are activated by FcRγ chain and subsequent downstream activation of MAPKs, PI3K, and PLC-γ. In order to establish if Syk activation via FcRs participates in T cell activation, we probed for Syk phosphorylation in the activation loop at Tyr525/526 in cells treated with ICs and sublytic MAC. First we prepared the immunoprecipitates using monoclonal anti-FcγRIIIa/b antibody from cells treated with sublytic MAC or ICs or in combination, when probed with anti-pSyk, showed phosphorylation of a protein band that migrated at 72 kD. This along with experiments presented in the earlier sections confirmed activation of Syk by synergistic action of ICs and sublytic MAC (FIG. 3C).

### T Cell Activation Evidenced by Tyrosine Phosphorylation of TCR Signaling Proteins:

Briefly, 1×10⁶ Jurkat cells were washed and suspended in 1 ml of RPMI and plated in 24 well culture plates. Cells were starved for 4 h in plain RPMI for 4 h prior to treatment. These cells were treated with purified MAC alone, purified ICs alone or combination of the IC with MAC. Purified pre-assembled MAC (2.5 μg) and ICs purified from two SLE patients at two concentrations, SLE-IC (1) at 1 μg and SLE-IC (2) at 4.5 μg were used. These cells were then incubated in a CO₂ incubator. At the end of the incubation period, cells were removed from the culture dish, washed with ice-cold PBS and lysed with buffer containing 20 mM Tris (pH8.0), 137 mM NaCl, 5 mM Na₂EDTA, 10% (v/v) Glycerol, 1% (v/v) Triton X-100, 1 mM EGTA, 10 mM sodium fluoride, 1 mM PMSF, 1 mM aprotinin, 1 mM leupeptin, and 10 mM Na₃ VO₄. The cells were vortexed and briefly sonicated, and then subjected to centrifugation at 13,500 rpm in a micro centrifuge (Eppendorf 5424). The protein quantities were estimated using a commercial protein assay kit (Bio-Rad, CA, USA) and a total of 50 μg protein was mixed with SDS-PAGE 4× loading buffer (Invitrogen) containing 50 mM DTT. The samples were electrophoresed using NuPAGE 4 to 12% gradient gel and transferred to immunobilon PVDF membrane (Millipore, USA). The blots were incubated with blocking buffer composed of 3% BSA at RT. Thereafter, blots were washed with Tris-buffered saline containing Tween-20 (TBS-T), and the membranes were probed with PY20 anti-phosphotyrosine monoclonal antibody followed by anti-mouse-HRP antibody. These experiments showed us that ICs and sublytic MAC triggered phosphorylation of a number of proteins, which based on the molecular mass were TCR associated proteins (FIG. 4A)

### Western Analysis of Lck, Syk, Src, and Zap-70:

In order to investigate the activation of the T cells, the membrane blots prepared as above and were probed for phospho-Lck (Tyr¹⁹²/Ser¹⁹⁴) (Santa Cruz Biotechnology, Santa Cruz, Calif.), phospho-Syk (Tyr^{525/526}), phospho-Src (Tyr⁴¹⁶), and phospho-Zap-70 (Tyr³¹⁹) (Cell Signaling Technologies, Boston, Mass.). We used primary antibodies at a dilution of 1:5000 in 3% BSA in TBS-T. Next the blots were probed with an appropriate dilution of the secondary antibodies conjugated to HRP enzyme. The signals were developed using a chemiluminescent substrate (Millipore, USA). The same blots were re-probed sequentially using anti-Syk, anti-Lck, anti-Src and anti-Zap-70. After Western blot analysis, the membranes were striped and stained with Commasie Blue R-250 stain, the stained membranes were scanned and images were analyzed using Image J software (NIH, USA) to compare the intensity of protein loading for each lane. These experiments showed us that the ICs and sublytic MAC triggered activation of TCR signaling pathway (FIG. 4B) (accepted for publication in Journal of Biological Chemistry).

Differentiation into FoxP3′ T Cells (iTregs):

The naïve CD4⁺ T cells were purified using human CD4⁺ naïve T cell isolation kit (Miltenyi Biotec, Germany). The cells were maintained in complete RPMI supplemented with 20 IU of IL-2 for five to seven days before the stimulation. Purified naïve CD4⁺ T cells (95% positive for CD4⁺ and CD45RA staining) at a density of 1×10⁶/ml were plated in 24 well tissue culture plates and kept in plain RPMI for 4 h. Then using 1 ml each, cells were treated with 2.5 μg of MAC or 1 μg of SLE-ICs or combination of both MAC and ICs for 2 h. Control cells did not receive any treatment. After treatment the media was replaced with complete media supplemented with 20 ng of IL-2 and cells were allowed to grow for 24 h. Thereafter, total of 100 ng of TGF-β1 was added to the cultures and cells were allowed to grow further. On day five the cells were harvested and analyzed for the expression of FoxP3 population using regulatory T cell kit from BD Biosciences (CA, USA) as per the protocol supplied by the manufacturer. The flow analysis was carried out using BD SLRII flow cytometer and data was analyzed using Flowjo (Treestar, Oreg.). The cells were initially gated on CD4, thereafter CD25⁺ and FoxP3⁺ expression was analyzed. We observed increase in the cell population that expressed FoxP3 upon treatment with ICs and sublytic MAC (FIG. 5A). In subsequent experiment when these experiments were carried out in the presence of anti-CD3 antibody, a similar but enhanced increase in the generation of FoxP3 expressing iTregs was observed (FIG. 5B).

### MODE OF CARRYING OUT THE INVENTION

What is described are the steps of process to carry out this invention.
1. Human naïve T cells are harvested from the peripheral blood lymphocytes that are obtained from blood collected in presence of anticoagulant.
2. Thereafter, the naïve T cells are isolated using positive selection with either anti-CD25 and/or anti-CD4⁺ coated beads.
3. The cells can also be isolated using a negative selection by adsorbing the monocytes, B cells, NK cells, γ and δ T cells.
4. The naïve T cells are grown in culture, expanded under appropriate conditions and phenotyped to establish them to be of naïve T cell phenotype.
5. These cells are treated using 0.1 μg to 10 μg IC, isolated from desired patient and sublytic MAC prepared in vitro by activation of the complement cascade using Zymogen A at a concentration of 1 μg to 10 μg for each 10×⁶ to 10×⁷ cells.
6. Naïve T cells also expanded using ICs and sublytic MAC in the presence of anti-CD3 from 0.1 μg to 10 μg/10⁶ cells
7. The treated cells are expanded in vitro cultures with conditions that allow them to expand in the presence of various combinations of cytokines to convert them into the effector T or regulatory T cells.
8. These expanded regulatory T cells at one to ten million in number can be infused into patient per kg of body weight.
9. In vivo the activation of T cells from ICs and sublytic MAC is altered by using a blocking agent that disrupts the synergistic action of these immune reactants to obtain a beneficial outcome in diseases caused by ICs and complement activation.

What is claimed is:

1. A method where human naïve CD4⁺ T cells are activated using the purified ICs at a concentration of 0.1 μg to 10 μg and 0.1 μg to 10 μg of MAC or TCC are used in combination.

2. A process where naïve CD4⁺ T cells require two signals, one from ICs and the other from MAC, according to claim 1, when grown in presence of cytokines become effector T cells

3. A process where naïve CD4⁺ T cells treated according to claim 1, in the presence of anti-CD3 antibody at a concentration of 0.1 μg to 10.0 μg to activate T cells from one to ten million cells.

4. A process to activate T cells according to claim 3, when grown in presence of appropriate cytokine generate regulatory T cells (iTreg).

5. A process according to claims 2 and 4 to generate iTreg cells for the treatment of patients with disorders arising from breakdown in immune tolerance. Such treatment can be achieved by generating T effector cells in vitro for subsequent in vivo use.

6. A method for modulation of the immune responses from ICs and sublytic MAC using an agent that disrupts the signaling cascade triggered by formation of ICs and MAC during inflammatory autoimmune diseases.

7. A use of a synthetic chemical, peptide, or biologics to disrupt the T cell activation as claimed in 6, to obtain a beneficial outcome for treatment of autoimmune inflammatory disorders.
